(19) United States
(12) Patent Application Publication
(10) Pub. No.: US 2022/0105464 A1
(43) Pub. Date: Apr. 7, 2022

(54) SYSTEMS AND METHODS FOR BIOMASS CARBON REMOVAL AND STORAGE

(71) Applicant: Donald Edward Holder, Stuart, FL (US)

(72) Inventor: Donald Edward Holder, Stuart, FL (US)

(21) Appl. No.: 17/209,318

(22) Filed: Mar. 23, 2021

Related U.S. Application Data
(60) Provisional application No. 63/087,192, filed on Oct. 3, 2020.

(51) Int. Cl. B01D 53/84; C12N 1/06; C12N 1/02; C12N 1/12; C12N 1/20

(52) U.S. Cl. CPC B01D 53/84; C12N 1/066; C12N 1/02; B01D 2257/504; C12N 1/20; B01D 2251/95; C12N 1/12

(57) ABSTRACT

A method for carbon capture and storage includes growing in water a biomass of photosynthetic microorganisms that capture carbon from a carbon source for growth; removing a portion of the biomass; and storing the removed biomass portion in an underground formation for carbon sequestration.

### CROSS-REFERENCE TO RELATED APPLICATIONS

This application claims the benefit of U.S. Provisional Patent Application Ser. No. 63/087,192, filed Oct. 3, 2020, the entirety of which is incorporated herein by reference.

### FIELD OF THE INVENTION

The present invention relates to reducing global warming through carbon sequestration and, more particularly, to systems and methods of using photosynthetic microorganisms to capture carbon and storing the microorganisms in underground formations to prevent the carbon from being released into the atmosphere as a greenhouse gas.

### BACKGROUND OF THE INVENTION

The increase of carbon dioxide in Earth's atmosphere is causing adverse conditions for various stakeholders of the planet. Carbon dioxide acts a greenhouse gas, resulting in the gradual warming and changing of climate. Experts have deemed this an important issue and have recommended that governments and corporations strive to limit atmospheric carbon dioxide levels below specifications.

Several regulations and pledges have been made to keep atmospheric levels of carbon dioxide below given limits. Many of the strategies being employed focus on green energy sources such as wind, water, and solar with the goal of reducing the combustion of fossil fuels. Carbon capture and storage is an alternative strategy which focuses on removing carbon dioxide from the air, or from a gas stream that exhausts into the air.

Photosynthesis is an organic process that uses light energy to create carbohydrates from carbon dioxide and water. Some microorganisms, such as microalgae, cyanobacteria, diatoms, and phytoplankton, possess photosynthetic capabilities, and are therefore able to remove carbon dioxide from the atmosphere. While the oxygen is typically released back into the air, the carbon is used by the organism and contributes to the overall biomass.

While it may be appealing to utilize the biomass for functional purposes, such as in food, fertilizer, or fuel—in all these cases the carbon ultimately returns to the atmosphere as a greenhouse gas. Accordingly, there is a need and desire for improved systems and methods for biomass carbon capture that does not return the carbon to the atmosphere as a greenhouse gas.

### SUMMARY OF THE INVENTION

An effective method for preventing the carbon from returning to the atmosphere is to move the biomass underground into a depleted oil or gas well for long term or permanent storage. This is fitting, as the increase in atmospheric carbon can be contributed to the oil and gas that was pulled out of these underground wells. Using or growing photosynthetic microorganisms to capture carbon from the atmosphere and then injecting and storing that biomass underground in depleted oil or gas wells is essentially putting it (the carbon) back where it came from.

Embodiments of the invention provide methods, systems, and apparatuses for capturing carbon dioxide using photosynthetic microorganisms, and then injecting said biomass in an underground opening, such as a well, cavern or mine, either occurring naturally or unnaturally.

In some embodiments, the microorganisms comprise cyanobacteria, diatoms, microalgae, phytoplankton, or euglena. Different organisms thrive in particular environments and climates, and therefore, the species selection should be chosen appropriately for the selected location.

In some embodiments, an apparatus for growing or farming the microorganism includes ponds, raceway ponds, bioreactors, or film belts; using light energy sourced from the sun or artificial light; using direct capture of carbon dioxide from the air or supplemented carbon dioxide from a carbon dioxide source such as an exhaust stream, a pressurized tank, or pressurized air bubbled into the water mixture.

In some embodiments, the water-microorganism mixture will undergo an agitation process comprising paddlewheels, pumps, jets, bubbling, mechanical stirring, or mixing.

In some embodiments, the water in which these organisms grow may be replenished by an external source, comprised of potable water, reclaimed water, sewage, wastewater, pit water, frack water, or underground natural sources. The water may be supplemented with added nutrients to promote growth.

In some embodiments, the ability to move and hold microorganism-water mixtures is comprised of transfer pumps and external tanks in order to raise or lower levels to mitigate rain or evaporation, or inoculate additional ponds, or provide water or bio samples for evaluation and measurements.

In some embodiments, the water in which these organisms grow will be supplemented by recycling water from the biomass-water mixture by a separation method, comprised of cyclone separation, centrifugation, filtration, settlement, flocculation, evaporation, distillation, elutriation, adsorption or scraping.

In some embodiments, the biomass will be transported to the underground access site by a transportation process comprised of pumped-through piping from a growth facility or tank delivery by vehicle.

In some embodiments, the biomass will be relocated underground by pressure or mechanical means, comprised of pumping, hydraulic force, pneumatic force, vacuum dragged, gravity driven, or injection.

In some embodiments, the underground formation, after injected to capacity with biomass, may be sealed by a process comprised of capping or mechanical sealing of the bore or exit paths or both.

In general, in one aspect, a method for carbon capture and storage is provided.

The method includes:
(a) growing in water a biomass of photosynthetic microorganisms that capture carbon from a carbon source for growth;
(b) removing a portion of the biomass; and
(c) storing the removed biomass portion in an underground formation for carbon sequestration.

In further aspects, the method may include the photosynthetic microorganisms being selected from one or more of cyanobacteria, diatoms, microalgae, phytoplankton, and euglena.

In further aspects, the method may include providing a habitat for the photosynthetic microorganism, and wherein in the growing step the photosynthetic microorganisms are grown in the habitat.

In further aspects, the habitat can be one or more of stagnant ponds, aerated ponds, raceway ponds, bioreactors, and film belts.

In further aspects, the carbon source can be one or more of direct air capture, introduction into the water by aeration, combustion exhaust, flue stream, and pressurized containers containing carbon dioxide gas.

In further aspects, the method can include lysing the microorganism cells before storing the biomass underground. Lysing can be performed by one or more of physical, thermal, hydraulic, sonic, enzymatic, osmotic, chemical, detergent, reagent, electricity, and freeze thaw, for example.

In still further aspects, the method can include dewatering the removed biomass portion to separate water from the removed biomass portion before storing in the underground formation. The dewatering can be performed by one or more of centrifuging, filtering, scraping, settling, evaporation, distillation, elutriation, flocculation, and adsorption, for example. The separated water dewatering can be returned to the water in which the biomass is grown, reclaimed, or discarded.

Numerous objects, features and advantages of the present invention will be readily apparent to those of ordinary skill in the art upon a reading of the following detailed description of presently preferred, but nonetheless illustrative, embodiments of the present invention when taken in conjunction with the accompanying drawings. The invention is capable of other embodiments and of being practiced and carried out in various ways. Also, it is to be understood that the phraseology and terminology employed herein are for the purpose of descriptions and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

For a better understanding of the invention, its operating advantages and the specific objects attained by its uses, reference should be had to the accompanying drawings and descriptive matter in which there are illustrated embodiments of the invention.

### BRIEF DESCRIPTION OF THE DRAWINGS

The following drawings illustrate by way of example and are included to provide further understanding of the invention for the purpose of illustrative discussion of the embodiments of the invention. No attempt is made to show structural details of the embodiments in more detail than is necessary for a fundamental understanding of the invention, the description taken with the drawings making apparent to those skilled in the art how the several forms of the invention may be embodied in practice. Identical reference numerals do not necessarily indicate an identical structure. Rather, the same reference numeral may be used to indicate a similar feature of a feature with similar functionality. In the drawings:

FIG. 1 is a diagrammatic view of a system for biomass carbon removal and storage in accordance with embodiments of the invention; and

FIG. 2 is a diagrammatic view of the system of FIG. 1 and showing another configuration of the system.

### DETAILED DESCRIPTION OF THE INVENTION

The invention is directed towards methods, systems, and apparatuses to capture carbon using photosynthetic microorganisms such as microalgae, diatoms, or cyanobacteria, and then relocating the produced biomass underground for storage to prevent releasing the captured carbon as greenhouse gas.

Embodiments of the invention provide for the removal of carbon dioxide, a greenhouse gas, from the air, or from an exhaust stream that will enter the air, and to storing the captured carbon underground. Carbon capture is performed by growing a biomass of photosynthetic microorganisms that remove carbon from carbon dioxide, releasing the resulting oxygen and committing the carbon atom to the growth and multiplication of the microorganism, resulting in a conversion from carbon dioxide into biomass growth. The method additionally involves transferring this biomass underground where it is stored, unable to be naturally reintroduced into the atmosphere.

In embodiments, a habitat is provided for the microorganisms to grow by providing water for a habitat, exposure to light energy, exposure to carbon dioxide, and access to nutrients.

In aspects, for effective carbon capture, supplement light energy and nutrients, such as those containing the elements of nitrogen, phosphorous, trace metals, and other essential nutrients may be provided. Additional light energy may be provided by artificial light sources.

In aspects, the method to grow the biomass includes a body of water, usually in a pond. This pond may be shaped in a raceway configuration to aid in circulation, aeration, and agitation. The agitation can be provided by pumps, paddles (paddlewheels), or jets that move the water to promote mixing. Mixing allows increased exposure to carbon dioxide, thermal mixing, and for deeper cells to reach the surface for equal light exposure. A belt system may be used to expose attached organisms directly to the air by circulating above the surface of the water.

The systems and methods disclosed here in are not limited to ponds. In embodiments, the biomass can be produced in bioreactors, on film belts, or can be filtered from natural sources containing the biomass.

The water to fill the body of water may be provided from a number of sources, including, but not limited to potable water, reclaimed water, sewage, wastewater, pit water, frack water, or underground natural sources. The water may also be supplemented with nutrients to grow the biomass. The most convenient may include water naturally occurring or previously injected into the ground. This water may be extracted from underground and provided to fill or supplement the body of water or make up for evaporation lost. Wastewater may also be utilized, with the added benefit of providing a useful purpose for the wastewater.

Supplemental carbon dioxide may be introduced by bubbling gas containing carbon dioxide into the body of water. This gas may be sourced from the exhaust of a combustion or chemical process, or from a pressurized tank.

As the biomass proliferates, a portion will be removed from the habitat and prepared for underground injection. This can be accomplished continuously or in batches. A volume of the water-microorganism mixture can be suctioned, pumped, or mechanically moved from the habitat through a system consisting of pipes or containers. The volume may be dewatered to recycle the water back into the habitat before injecting the biomass underground. The volume could be dewatered, for example, by centrifuging, filtering, scraping, settling, evaporation, distillation, elutriation, flocculation, or adsorption methods.

The biomass is then injected underground into an oil well, gas well, cavern or mine by pumping, injecting, pneumatic forcing, hydraulic forcing, or other state of the art methods. Once the well is at capacity or at a point where no more biomass will be added, the injection bore, or access path may be capped or sealed to prevent escape of the captured carbon.

In aspects, the microorganism cells can be subject to lysing by physical, thermal, hydraulic, sonic, enzymatic, osmotic, chemical means or by use of detergent, reagent, electricity, or freeze thaw, for example, before being injected underground.

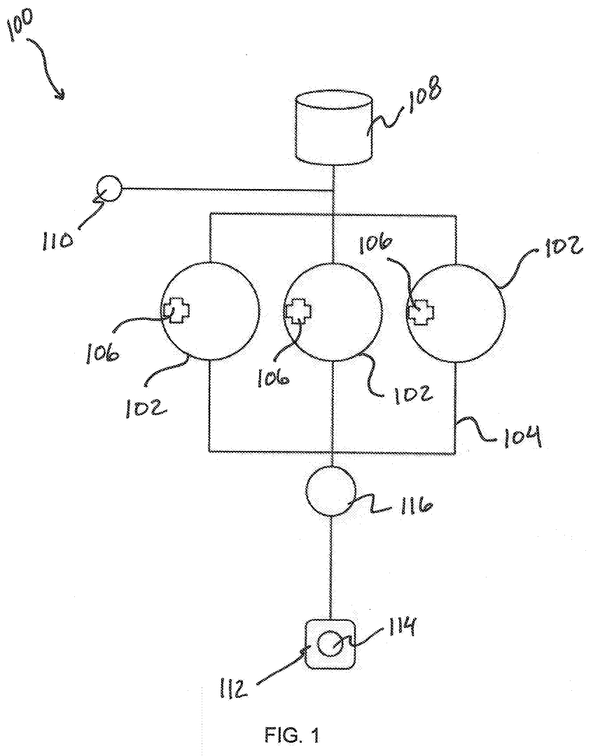

In FIG. 1 there is shown diagrammatically a system 100 in accordance with embodiments of the invention for implementing biomass carbon removal and storage. As depicted, the system 100 includes a plurality of ponds 102, such as, for example raceway ponds. Ponds 102 are used to hold the microorganisms and to the grow the biomass.

The ponds 102 are connected by a plumbing system 104, representatively shown. The plumbing system 104 can have pumps, valves, and other equipment necessary to control flow into and out of the ponds 102. One or more ponds 102 can be fitted with one or more agitators 106, such as, for example paddlewheel agitators for agitating the water and biomass held in the pond.

The ponds 102 may be connected to a holding tank 108. The ponds 102 may be connected to an external supply of water 110, such as, for example, a water well for supplying water to the ponds.

The ponds 102 are also connected to an injection pump 112 that is connected to an injection wellhead 114 that is connected to an underground formation by one or more conduits for injecting biomass from the ponds into the underground formation through the wellhead. A dewatering unit 116, such as, for example, can be connected to the ponds 102 before the wellhead 114 and used to dewater biomass before being injected into the underground formation.

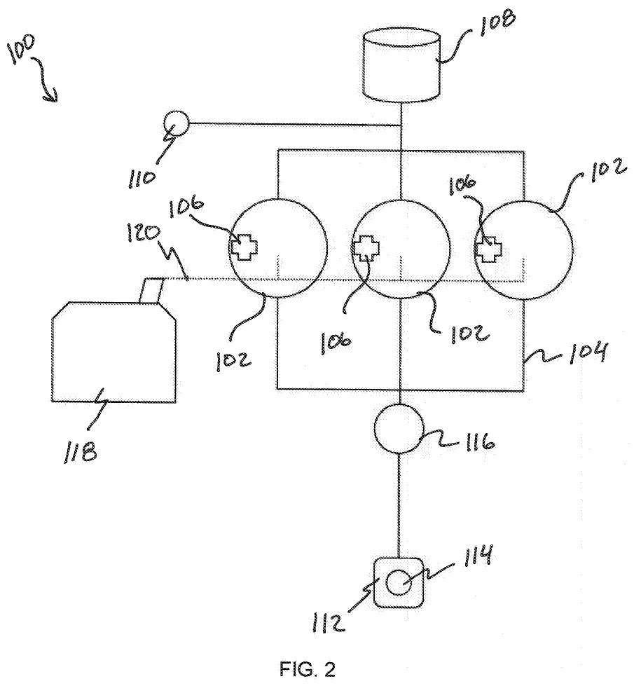

In FIG. 2, there shown system 100 in accordance with embodiments of the invention for implementing biomass carbon removal and storage. As depicted, system 100 further includes a connection to a carbon dioxide (CO2) source 118 for receiving CO2 therefrom for conversion into biomass by the microorganisms. The CO2 received from the source 118 can be in the form of CO2 gas, or combustion gas containing CO2, for example. The ponds 102 can be connected to source 118 so that source is injected or bubbled or otherwise introduced to the microorganisms held in the ponds 102 via line 120.

It is to be understood that the specific configuration of system 100 is representative only, as the system could be configured in numerous ways and remain within the scope of the embodiments of the invention. Accordingly, the invention should not be construed to be limited to the specific system that is representatively shown in FIGS. 1 and 2. There are numerous was the invention could be implemented without departing from the spirit and scope of this disclosure.

While the invention has been particularly shown and described with respect to the illustrated embodiments thereof, it will be understood by those skilled in the art that the foregoing and other changes in form and details may be made therein without departing from the spirit and scope of the invention.

What is claimed is:

1. A method for carbon capture and storage, comprising:
growing in water a biomass of photosynthetic microorganisms that capture carbon from a carbon source for growth;
removing a portion of the biomass; and
storing the removed biomass portion in an underground formation for carbon sequestration.

2. The method of claim 1, wherein the photosynthetic microorganisms are selected from one or more of cyanobacteria, diatoms, microalgae, phytoplankton, and euglena.

3. The method of claim 1, further comprising:
providing a habitat for the photosynthetic microorganism, and wherein in the growing step the photosynthetic microorganisms are grown in the habitat.

4. The method of claim 3, wherein the habitat is selected from one or more of stagnant ponds, aerated ponds, raceway ponds, bioreactors, and film belts.

5. The method of claim 1, further comprising:
agitating the water containing the biomass.

6. The method of claim 5, wherein in the agitating step, agitation is performed by one or more of paddlewheels, pumps, water jets, bubbling, mechanical stirring, and mixing.

7. The method of claim 1, wherein the carbon source is one or more of direct air capture, introduction into the water by aeration, combustion exhaust, flue stream, and pressurized containers containing carbon dioxide gas.

8. The method of claim 1, further comprising:
lysing the microorganism cells before storing the biomass underground.

9. The method of claim 8, wherein the lysing is performed by one or more of physical, thermal, hydraulic, sonic, enzymatic, osmotic, chemical, detergent, reagent, electricity, and freeze thaw.

10. The method of claim 1, wherein in the underground formation is a natural or man-made underground formation.

11. The method of claim 1, further comprising:
dewatering the removed biomass portion to separate water from the removed biomass portion before storing in the underground formation.

12. The method of claim 11, wherein in the dewatering step, dewatering is performed by one or more of centrifuging, filtering, scraping, settling, evaporation, distillation, elutriation, flocculation, and adsorption.

13. The method of claim 11, wherein the separated water during dewatering is returned to the water in which the biomass is grown.

14. The method of claim 11, wherein the separated water during dewatering is reclaimed or discarded.

15. The method of claim 1, wherein in the removal step is performed continuously or periodically.
